(19) United States
(12) Patent Application Publication
(10) Pub. No.: US 2011/0028839 A1
(43) Pub. Date: Feb. 3, 2011

(54) Acoustic Sensor

(76) Inventor: John P. Keady, Fairfax Station, VA (US)

Correspondence Address: John P . Keady, 138 Crestridge Rd., Fairfax Station, VA, 22039, US

(21) Appl. No.: 12/849,011

(22) Filed: Aug. 2, 2010

Related U.S. Application Data
(60) Provisional application No. 61/230,159, filed on Jul. 31, 2009.

(51) Int. Cl. A61B 8/00; B06B 1/00

(52) U.S. Cl. 600/437; 367/140

(57) ABSTRACT

At least one exemplary embodiment is directed to an acoustic sensor, that can be used to identify various fluids as well as concentrations of compounds dissolved in fluids.

### CROSS-REFERENCE TO RELATED APPLICATIONS

This application claims the benefit of U.S. provisional patent application No. 61/230,159 filed 31 Jul. 20098. The disclosure of which is incorporated herein by reference in its entirety.

### FIELD OF INVENTION

The present invention is relates to sensor technology, and more particularly to an acoustic sensor that can be used to identify and monitor fluid environments.

### BACKGROUND

The Acoustic Sensor uses acoustic waves traveling through a medium to identify the medium. The reflected and transmitted waves can be used to not only detect a fluid (in the scientific community fluid is a general term for both liquids and gases) but to also determine the fluid composition (e.g., pure water, salt water, oil, alcohol, air). Additionally the information can be used to determine some of the medium's state (e.g., pressure and temperature).

The Acoustic sensor is useful in industrial markets (e.g., flow monitoring), scientific markets (e.g., Earth science measurement probes for example to monitor salinity, Space Planetary probes to detect various fluids), military markets (e.g., to identify liquids quickly and monitor military systems), government markets (e.g., airport fluid screening, border control screening, law enforcement), and medical markets (blood flow diagnostics).

The sensor market has an established market and an unestablished market. The basic market categories include flow monitoring and measurement, fluid detection, fluid identification, pressure and temperature measurement. The established markets are Insitu pipe flow and pressure change monitoring, as well as fluid identification and monitoring for scientific probes (e.g., buoy salinity measurements). The unestablished market includes airport screening of liquids, military screening of liquids, and scientific experimental monitoring of flow characteristics (e.g., rocket and aircraft engine injector design downstream mixing measurements).

Technical Review

There exist several sensor technologies, of various levels of complexity, to identify substances (e.g., solids, liquids, and gases). The sensors have various advantages and disadvantages with regards to power usage, complexity of design, reliability, ease of use, real time identification, size, minimal sampling size, minimal sample time, and calibration complexity to name a few.

Many fluid (liquid and gas) sensors, require destruction of the sample (e.g., a mass spectrometer), and some others require optical sources and detection systems (e.g., spectrometer). Few are constructed to be versatile enough to sample either a stagnant system (no flow velocity) or a mobile system.

Basic Acoustic Physics

Basically when an acoustic wave is incident upon and travels through a medium its wave properties changes (e.g., spectrum, phase, transmitted intensity, reflected and absorbed intensities). More specifically wave properties change with a medium's impedance, which is itself a function of the path length through the medium, the medium's composition, and the medium's temperature and pressure.

Known wave properties incidence on a medium can be used to identify the medium and its state (e.g., pressure, temperature).

Additionally if the medium is moving while the acoustic wave travels through the medium, Doppler shifting of the measured transmitted spectrum can provide a measure of the velocity of the medium.

### SUMMARY

At least one exemplary embodiment is directed to an acoustic sensor comprising: a first receiver configured to emit a first acoustic signal; a first microphone configured to measure a first measurement signal; a second microphone configured to measure a second measurement signal; a chamber; and a first medium, where the first medium is in the chamber, where at least a first portion of the first acoustic signal passes through the medium, where the first measurement signal includes at least a second portion of the first acoustic signal, where the second measurement signal includes at least a third portion of the first acoustic signal, where the second and third portions are compared to stored identification data, where a match of the second and third portions with the stored identification data produces an identification for the first medium.

At least one exemplary embodiment is directed to a medical diagnostic device comprising: a first receiver configured to emit a first acoustic signal; a first microphone configured to measure a first measurement signal; a second microphone configured to measure a second measurement signal; and a finger where at least a first portion of the first acoustic signal passes through the finger, where the first measurement signal includes at least a second portion of the first acoustic signal, where the second measurement signal includes at least a third portion of the first acoustic signal, where the second and third portions are compared to stored identification data, where a match of the second and third portions with the stored identification data produces an identification for the blood sugar levels in the finger.

Embodiments of the invention are directed to a method and system for sound monitoring, measuring, reporting, and providing over a network using mobile devices. Sound reports can be generated that associate sound levels with a time and a location. The sound reports can then be shared with other network users.

### BRIEF DESCRIPTION OF THE DRAWINGS

Exemplary embodiments of present invention will become more fully understood from the detailed description and the accompanying drawings, wherein:

FIG. 1a illustrates a general model of an anechoically terminated tube referred to herein also as an impedance tunnel;

FIG. 1b illustrates the general configuration of an anechoically terminated tube;

FIG. 1 illustrates a general configuration of an acoustic sensor;

FIG. 2A illustrates an unoccluded intensity profile, while FIG. 2B illustrates an occluded intensity profile, illustrating the capacity to distinguish between two occluding fluids 220 and 230,

FIG. 3 illustrates another acoustic sensor in accordance with at least one exemplary embodiment,

FIGS. 4A-6 illustrates other configurations in accordance with at least one exemplary embodiment of an acoustic sensor, for example FIG. 5 illustrates a cross sectional view of a finger pressing against a speaker source port 110, and two or more analysis microphones 183, 185, and 187;

FIGS. 7-9 illustrate relative acoustic amplitude (e.g., sound pressure level) as a function of frequency for water at 0.5 grams of NaCl in 100 ml water, 1.0 gram of NaCl in 100 ml water, sea water, water with 1.0 grams of sucrose, and water as measured by the upstream microphone (UM), as a function of reservoir pressure of 0.25 bar, 0.3 bar, and 0.35 bar respectively (gauge pressure);

FIG. 10 illustrates the relative difference (compared with distilled water) sound amplitudes (dB) as a function of frequency between a concentration of NaCl of 0.5 grams in 100 ml, and NaCl of 1 gram in 100 ml water;

FIG. 11 illustrates the relative differences of amplitude of 1 gram of NaCl solution, alcohol, baby oil, sea water, and 1 gram sugar in 100 ml, at 0.35 bar gauge pressure;

FIG. 12 illustrates the relative differences of amplitude of 1 gram of NaCl solution, air, alcohol, baby oil, and 1 gram sugar in 100 ml, at 0.35 bar gauge pressure;

FIG. 13 illustrates the complex transfer function between the upstream and downstream microphone for alcohol at three pressures 0.25 bar, 0.3 bar, and 0.35 bar;

FIG. 14 illustrates the complex transfer function between the upstream and downstream microphone for air at three pressures 0.25 bar, 0.3 bar, and 0.35 bar;

FIG. 15-17 illustrate spectrograms for 1 gram of sucrose in 100 ml water at three pressures 0.25 bar, 0.3 bar, and 0.35 bar;

FIG. 18-20 illustrate spectrograms for 1 gram of sucrose, 0.5 grams of NaCl in 100 ml, and 1 gram of NaCl in 100 ml at the same pressure 0.25 bar gauge; and

FIGS. 21-23 illustrate NaCl various concentrations and sucrose various concentrations, where the coherence between the upstream and downstream microphone are illustrated, where differences in concentration can be clearly seen and differences in substance can be seen, and where the concentration differences occur in a frequency bands and where the substance differences occur in the same and other frequency bands.

### DETAILED DESCRIPTION

The following description of exemplary embodiment(s) is merely illustrative in nature and is in no way intended to limit the invention, its application, or uses.

Processes, techniques, apparatus, and materials as known by one of ordinary skill in the art may not be discussed in detail but are intended to be part of the enabling description where appropriate. For example specific computer code may not be listed for achieving each of the steps discussed, however one of ordinary skill would be able, without undo experimentation, to write such code given the enabling disclosure herein. Such code is intended to fall within the scope of at least one exemplary embodiment.

Notice that similar reference numerals and letters refer to similar items in the following figures, and thus once an item is defined in one figure, it may not be discussed or further defined in the following figures.

In all of the examples illustrated and discussed herein, any specific values, should be interpreted to be illustrative only and non-limiting. Thus, other examples of the exemplary embodiments could have different values.

While the specification concludes with claims defining the features of the embodiments of the invention that are regarded as novel, it is believed that the method, system, and other embodiments will be better understood from a consideration of the following description in conjunction with the drawing figures.

Advantage of Acoustic Sensors

Insitu measurements typically require physical sampling and hence disruption of the system measured. Electromagnetic (EM) based systems have been used for local property determination without physical sampling. Acoustic Wave (AW) based systems are less expensive and provide the same level of non physical sampling as EM systems without the more expensive EM source and detection devices. Additionally AW systems provide more reflection and transmission information that can be used to further refine fluid and state identification.

Introduction:

An impedance identification device is a piece of equipment designed to examine properties associated with a device occluding (sealing) the identification device at some position along its length. The device can include a medium to identify. The medium in the device (e.g., fluid in a tube) influences acoustic waves traveling through the medium, uniquely enabling the identification of the medium. The identification device is designed to impinge upon the device an incident acoustic signal, and to minimize any transmitted acoustic signal (i.e., through the device) from reflecting from the rear of the identification device.

Optimally the incident acoustic signal is a plane wave, which is partially reflected (i.e., reflected acoustic signal) at the boundary of the identification device medium and the device surface. Within the device standing waves may be generated as well, with a portion of the incident acoustic signal being transmitted through the device (i.e. transmitted acoustic signal) and traveling down the length of the identification device. At the end of the identification device a portion of the transmitted wave is reflected back toward the device (i.e. end reflected acoustic signal). Any acoustic source wave can be used as the acoustic probe source.

The identification device is designed to reduce the end reflected acoustic signal intensity by at least 30 dB from the unoccluded identification device at the location of occlusion.

If working properly the Impedance Identification device can provide data from which several properties can be calculated (e.g., reflection coefficient, transmission coefficient, absorption coefficient, device impedance, insertion loss through the device, and transmission loss through the device). These properties can be used to identify the sample. For example the coherence between two microphones at particular frequencies changes depending upon the material in the sample chamber. For example if water is sampled, various levels of NaCl, sucrose, and glucose can be identified.

Theory:

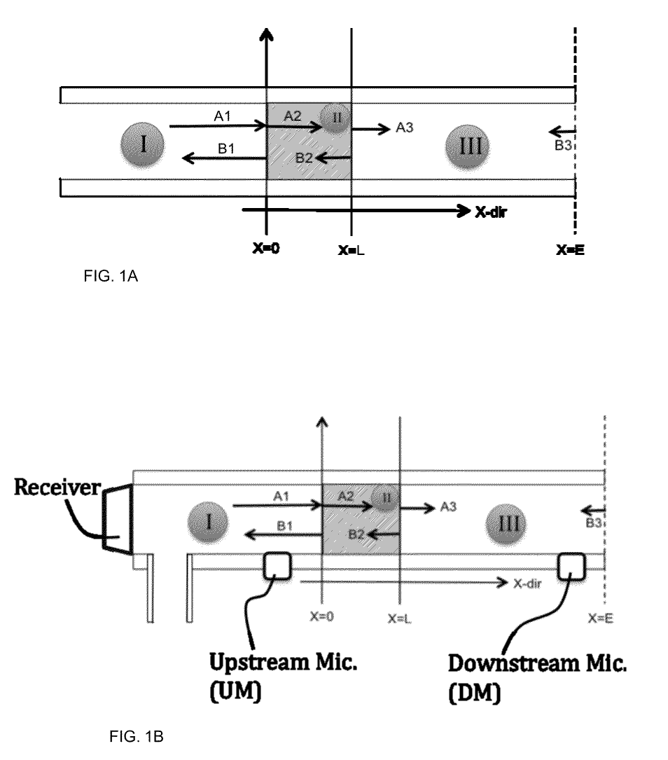

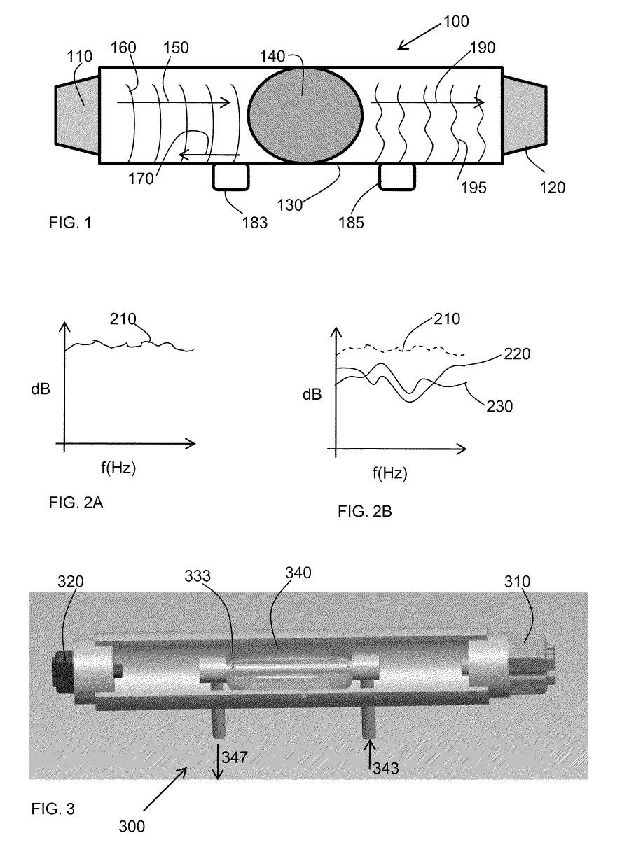

FIG. 1A illustrates the basic configuration of an occluding device (Region II) in an identification device environment.

A1, A2, A3, B1, B2, and B3 are magnitudes of time-harmonic waves. A pressure wave traveling from left to right (associated with A1) encounters the interface at x=0 where a portion is transmitted (associated with A2) and a portion is reflected (associated with B1). The transmitted pressure wave (associated with A2) has a portion reflected from the second interface x=L (associated with B2), and so on between the first and second interfaces setting of a standing wave in region II (not shown). The transmitted pressure wave in region II (associated with A2) will have a portion transmitted into region III (associated with A3). The pressure wave transmitted in region III will have a portion reflected from the end of the identification device at x=E (associated with B3). For purposes of discussion B3 will be ignored and in identification device design is intended to be at least 30 dB below A1 when B3 returns to the vicinity of X−0.

Deriving the equations governing the phenomena requires interface conditions to link regions. We use continuity of pressure and particle velocity at the interfaces (x=0, and x=L).

Derivation of Transmission (T) and Reflection (R) coefficients for the simplified model of FIG. 1A:

The particle velocity, u, can be related to impedance Z=ρc, and the pressure, p, by equations (1) and (2). Note that in general a pressure wave can be expressed as P=P₀e^{i(ωt-kx)}, for equations (1) through (13) the time varying portion will be ignored since it will fall on both sides of equations (7) to (10) and thus cancel out.

p=Zu for a forward traveling waves (1)

p=−Zu for a backward traveling waves (2)

The forward (the incident wave, from the left) and backward (the reflected wave) in region 1 can be expressed as:

P1=A1e^{-ik1x}+B1e^{ik1x} (3)

Likewise the forward (the incident wave, from the left) and backward (the reflected wave) in region 2 can be expressed as:

P2=A2e^{-ik1x}+B2e^{ik1x} (4)

Finally the forward (the incident wave, from the left) and backward (the reflected wave) in region 3 can be expressed as:

P3=A3e^{-ik1(x-L)}+B3e^{ik1(x-L)} (5)

If the identification device is designed correctly then B3 is minimized (30 dB below A3) so that as an additional approximation:

P3˜A3e^{-ik1(x-L)}

The interface boundary conditions are expressed in equations (7) and (8) for the location x=0.

A1+B1=A2+B2 Interface Boundary Condition: continuity of pressure (7)

A1−B1=(Z1/Z2)(A2−B2) Interface Boundary Condition: continuity of particle velocity (8)

Likewise the interface boundary conditions are expressed in equations (9) and (10) for the location x=L.

A2e^{-ik1L}+B2e^{ik1L}=A3 Interface Boundary Condition: continuity of pressure (9)

A2e^{-ik1L}−B2e^{ikiL}=(Z2/Z3) A3 Interface Boundary Condition: continuity of particle velocity (10)

The transmission coefficient T and reflection coefficient R are related to the magnitudes of the harmonic waves and can be expressed by equations (11) and (13). Where special conditions of these equations can be used to help understand the phenomena that develops.

T=A3/A1=2/[(1+Z1/Z3)cos(k2L)+i(Z2/Z3+Z1/Z2)sin(k2L)] (11)

letΔ=[(1+Z1/Z3)cos(k2L)+i(Z2/Z3+Z1/Z2)sin(k2L)] (12)

R=B1/A1=[(1−Z1/Z3)cos(k2L)+i(Z2/Z3−Z1/Z2)sin(k2L)]/Δ (13)

In addition to transmission and reflection there is also absorption and dispersion. Absorption is the loss of energy, and dispersion the variation of a spectrum based upon a frequency dependency of the speed of sound. Absorption and dispersion will be discussed after a short discussion of special cases for T and R, that can lend help in analyzing the frequency response of data.

### Special Conditions of T and R

CASE 1: k2L=nπ, Z1=Z3, then perfect transmission and no reflection.

T=A3/A1=(−1)ⁿ2/[(1+Z1/Z3)] (11A)

letΔ=(−1)ⁿ[(1+Z1/Z3)] (12A)

R=B1/A1=[(−1)ⁿ(1−Z1/Z3)]/[(−1)ⁿ[(1+Z1/Z3)]]=(1−Z1/Z3)/(1+Z1/Z3) (13A)

If Z1=Z3 then we have:

T=A3/A1=(−1)ⁿ (11B)

letΔ=(−1)ⁿ2 (12)

R=B1/A1=0 (13B)

Example for L=10 mm, at a frequency of about 17150 Hz we would expect a peak in the downstream microphone intensity.

CASE2: k2L=(n−1/2)π, Z2=(Z1Z3)^{0.5}, then no reflection

T=A3/A1=2i(−1)ⁿ/[Z2/Z3+Z1/Z2] (11C)

letΔ=i(−1)ⁿ(Z2/Z3+Z1/Z2) (12C)

R=B1/A1=i(−1)ⁿ(Z2/Z3−Z1/Z2)/i(−1)ⁿ(Z2/Z3+Z1/Z2) (13C)

If Z2=(Z1Z3)^{0.5} then we have:

T=A3/A1=i(−1)ⁿ(Z3/Z1)^{0.5} (11D)

R=B1/A1=0 (13D)

Example for L=10 mm, at a frequency of about 8575 Hz we would expect a peak in the downstream microphone intensity if Z2=(Z1Z3)^{0.5}.

CASE3: k2L<<1, thus cos(k2L)˜1, and sin(k2L)˜k2L,

T=A3/A1=2/[(1+Z1/Z3)1+i(Z2/Z3+Z1/Z2)k2L] (11E)

letΔ=[(1+Z1/Z3)1)+i(Z2/Z3+Z1/Z2)k2L] (12E)

R=B1/A1=[(1−Z1/Z3)1+i(Z2/Z3−Z1/Z2)k2L]/Δ (13E)

For Z1=Z3 we have:

T=A3/A1=2/[2+i(Z2/Z3+Z1/Z2)k2L] (11F)

letΔ=[2+i(Z2/Z3+Z1/Z2)k2L] (12F)

R=B1/A1=i[(Z2/Z3−Z1/Z2)k2L]/Δ0 (13F)

CASE3A: L=10 mm, for f=100 Hz k2=2π/λ˜0.0018 mm⁻¹; k2L=0.018

T=A3/A1=2/[2+i(Z2/Z3+Z1/Z2)k2L]˜1 (11G)

R=B1/A1=i[(Z2/Z3−Z1/Z2)k2L]/Δ˜0 (13G)

CASE3B: L=10 mm, for f=20000 Hz k2=2π/λ˜0.3694 mm⁻¹; k2L=3.694

T=A3/A1=2/[2+i(Z2/Z3+Z1/Z2)3.694] (11H)

letΔ=[2+i(Z2/Z3+Z1/Z2)3.694] (12H)

R=B1/A1=i[(Z2/Z3−Z1/Z2)3.694]/Δ (13H)

Thus at the lower frequencies (e.g.,100 Hz) we would expect from the simple model to see less reflectivity and more transmission, while at higher frequencies (e.g., 20000 Hz) a mix of transmission and reflectivity.

### Absorption for the simplified model of FIG. 1A

As discussed the pressure can be expressed as P=P₀e^{i(ωt-kx)} in general the wave number k is also complex, giving

k=β−iα (14)

The pressure can be expressed then as:

P=P₀e^{i(ωt-kx)}=P₀e^{-αx}e^{i(ωt-Δx)} (15)

The term e^{-αx} reduces the amplitude over time, and α is commonly referred to as the absorption coefficient. Hence the amplitude A2 in the model above reduces in time based upon x and α, where α is typically frequency dependent, α(ω), where α and ω are related by a dispersion relationship (where dispersion results from a frequency dependency of the speed o sound). The coefficient α can be related to the phase velocity of the wave by:

cₚₕₐₛₑ=ω/β (16)

Note that α and β are not independent, but this will not be examined herein. The basic mechanisms for energy absorption are viscosity, heat conduction, relaxation, and boundary layer effects.

TABLE 1
The various frequency (f) dependencies of each on α(ω),

| | |
|---|---|
| Viscosity | α∝ f² |
| Heat Conduction | α∝ f² |
| Relaxation | α∝ f²/(f² + f²ᵣ) |
| Boundary-layer effects | (f)^{0.5} |

Note that relaxation is related to changing equilibrium conditions of a medium when temperature or pressure changes. For example if temperature changes the water absorption into air may change affecting the speed of sound and other properties. fᵣ is called the relaxation frequency and is related to the relaxation time τ of a change in condition, for example the relaxation time of oxygen vibration is 10⁻⁵ s. Thus measurement of the change of absorption as a function of pressure and/or temperature change can further identify chemical composition of a medium.

### Summary of Sound Absorption in Fluids¹

The relationships for the absorption coefficient are expressed below with a low frequency assumption (that the frequency is less than 50 MHz for air and less than 10000 MHz for water, which is well within the range of any acoustic data obtained in the identification device, which will typically fall below 20 kHz).
For viscosity the absorption coefficient is:

α=ημω²/2ρ₀c³₀ (17) due to viscosity

where the viscosity number, η, can be expressed as:

η=4/3+μ_{B}/μ, (18)

where μ_{B} is the bulk viscosity and μ is the shear viscosity coefficients. For a thermally conductive fluid the absorption coefficient is:

α=(γ−1)κω²/2ρ₀c³₀Cₚ due to a thermally conductive fluid (19)

where the γ is the ratio of specific heats Cᵥ/Cₚ and κ is the heat conduction coefficient.
For a thermoviscous fluid where the fluid is both viscous (17) and thermally conductive (19) the absorption coefficient is:

α=[μω²/2ρ₀c³₀](η+(1−γ)/Pr) due to a thermoviscous fluid (20)

where the Pr is expressed as:

Pr=μCₚ/κ. (21)

Note that the classical thermoviscous fluid absorption coefficient often ignores the μ_{B}/μ however this term can be relatively important for example for air the ratio can be about 0.6¹
For a relaxing fluid the absorption coefficient is:

α=mτω²/2c₀(1+ω²τ²) due to a relaxing fluid (22)

where the m can be expressed as:

m=(c²₀−c²_{∞})/c²₀ (23)

Note that cₒ is referred to as the equilibrium sound speed (where ωτ goes to 0) and c_{∞}, is referred to as the frozen sound speed (where ωτ goes to ∞) At low frequencies cₒ is the phase velocity, at high frequencies c_{∞}is the phase velocity.
For an example see pg. 321 of Blackstock, where there is a discussion how the relaxation absorption is the dominant absorption mechanism in air in the audio and low ultrasonic frequency region.
For Boundary Layer Absorption (in a round tube of radius a) in thermoviscous fluids the absorption coefficient is:

α=a⁻¹[μω/2ρ₀c²₀]^{0.5}(1+(γ−1)/(Pr)^{0.5}) due to Boundary Layer Absorption (in a round tube of radius a). (24)

The last topic, which aids in understanding an oscillatory occluding devices (e.g., one of flexible material) describes resonance frequencies of a cavity fluid model. Note that bipolar pulsating spheres, pressure release spheres, hollow spheres are treated in Blackstock pages 349-356 and lend insight into various oscillatory systems. A simple calculation of the eigenfrequencies reveals large eigenfrequencies (for example for the pressure release sphere described on page 354, for a=5 mm, c₀=343 m/sec, I=1, f₁₀₀=34,300 Hz). In this example one would expect to see a standing wave internal to the pressure release sphere, where on one of the frequencies of the standing wave is about 34,300 Hz.

### Summary of Time-Harmonic Finite Monopole

The mechanical impedance at any point r>=a for a pulsating sphere of average radius a (i.e., average because the surface is pulsating) can be expressed as:

Z_{mech}=ρ₀c₀[(k²r²/(1+k²r²))+i(kr/(1+k²r²))] (25)

CASE 4: r=a; ka>>1, large sources and/or high frequencies

Z_{mech}=ρ₀c₀4πa² (25A)

Note that the real value of 21A is akin to a purely resistive load suggesting efficient energy transfer into the surrounding fluid.
CASE 5: r=a; ka<<1, very small sources and/or low frequencies

Z_{mech}=iω4πa³ρ₀ (25B)

Note that the imaginary value of 21B is akin to a purely reactive load suggesting no acoustic power transfer into the surrounding fluid.

### Calculating Resonance Frequency of a Bubble in Liquid

Generally setting the impedance to zero allows one to solve for the resonance frequency. The resonance frequency provides us with insight into standing waves that can be set up in eth occluding device or in a sealed cavity created by the occlusion device (e.g., occlusion effect frequencies). The simple example of an air cavity in a fluid environment provides some insight into a general formula that can be used to calculate a resonance frequency that can be compared with results.
In general an acoustic impedance Z_{ac} can be expressed as a combination of an acoustic mass term M_{ac} (mass of fluid displaced) and the acoustic compliance C_{ac} of the gas volume created in a collapsing and expanding oscillating bubble.

Z_{ac}=iωM_{ac}+1/iωC_{ac} (26)

For resonance set Z_{ac}=0.
For an air cavity that is created the acoustic mass is:

M_{ac}=ρ₀/4πa (27)

while the acoustic compliance is:

C_{ac}=4πa³/3γP_{og}, where P_{og} is the static pressure of the gas inside the cavity. (28)

Thus (22) becomes:

Z_{ac}=iωρ₀/4πa+3γP_{og}/iω4πa³ (29)

setting (25) to 0 one gets a resonance frequency f₀ that can be expressed as:

f₀=(1/2π)(3γP_{og}/ρ₀a²)^{0.5} (30)

For example for a 10 mm diameter cavity where a=5 mm, γ=1.4 for air, ρ₀=1000 Kg/m³ for water, and P_{og} of about 1 atm or 101300 Pa of the gas, one obtains f₀=656.9 Hz. This is near occlusion effect frequencies seen in the literature and helps to lend some insight into basic processes.

### Identification Device Description:

The impedance identification device can be constructed so that B3 is at least 30 dB below A3 in intensity, although such a design is optional only.
FIG. 1B illustrates the general configuration of the impedance identification device.

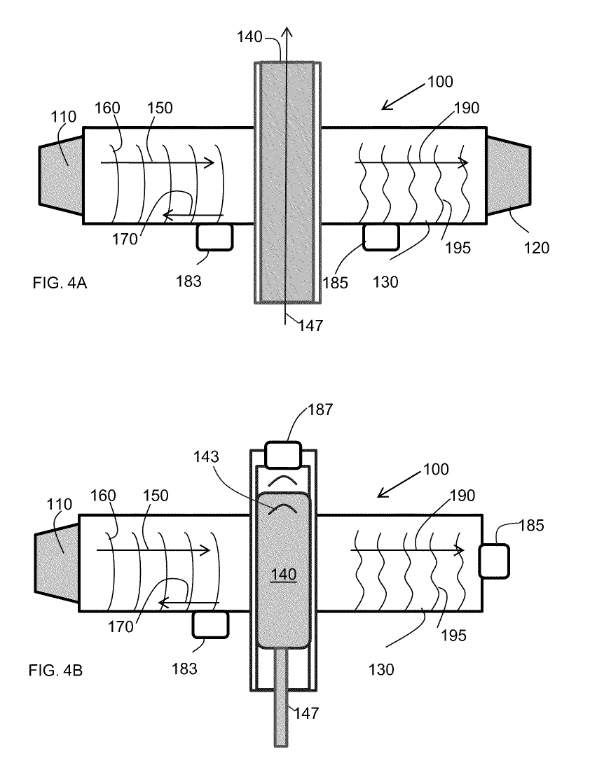

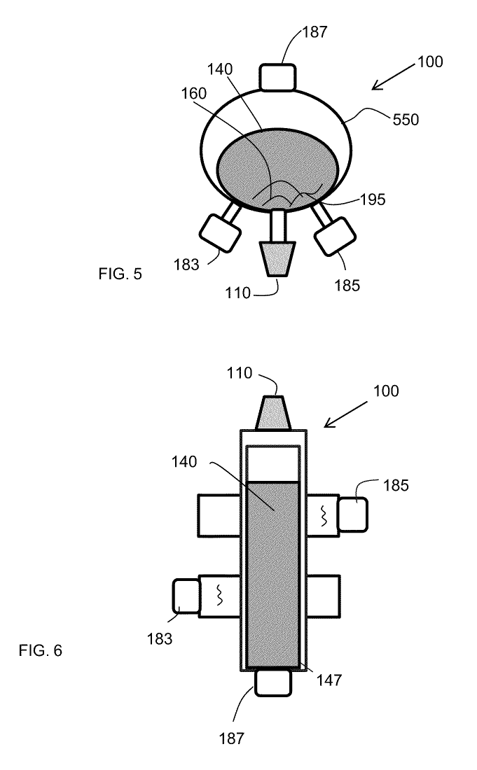

FIGS. 4A-6 illustrates other configurations in accordance with at least one exemplary embodiment of an acoustic sensor, for example FIG. 5 illustrates a cross sectional view of a finger pressing against a speaker source port 110, and two or more analysis microphones 183, 185, and 187;

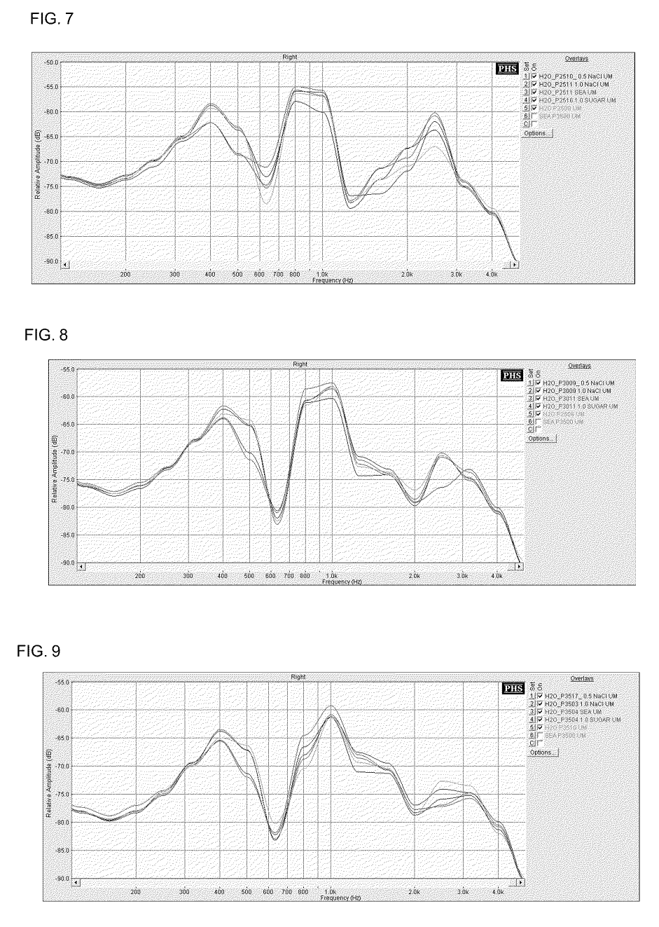

FIGS. 7-9 illustrate relative acoustic amplitude (e.g., sound pressure level) as a function of frequency for water at 0.5 grams of NaCl in 100 ml water, 1.0 gram of NaCl in 100 ml water, sea water, water with 1.0 grams of sucrose, and water as measured by the upstream microphone (UM), as a function of reservoir pressure of 0.25 bar, 0.3 bar, and 0.35 bar respectively (gauge pressure);

FIG. 10 illustrates the relative difference (compared with distilled water) sound amplitudes (dB) as a function of frequency between a concentration of NaCl of 0.5 grams in 100 ml, and NaCl of 1 gram in 100 ml water;

FIG. 11 illustrates the relative differences of amplitude of 1 gram of NaCl solution, alcohol, baby oil, sea water, and 1 gram sugar in 100 ml, at 0.35 bar gauge pressure;

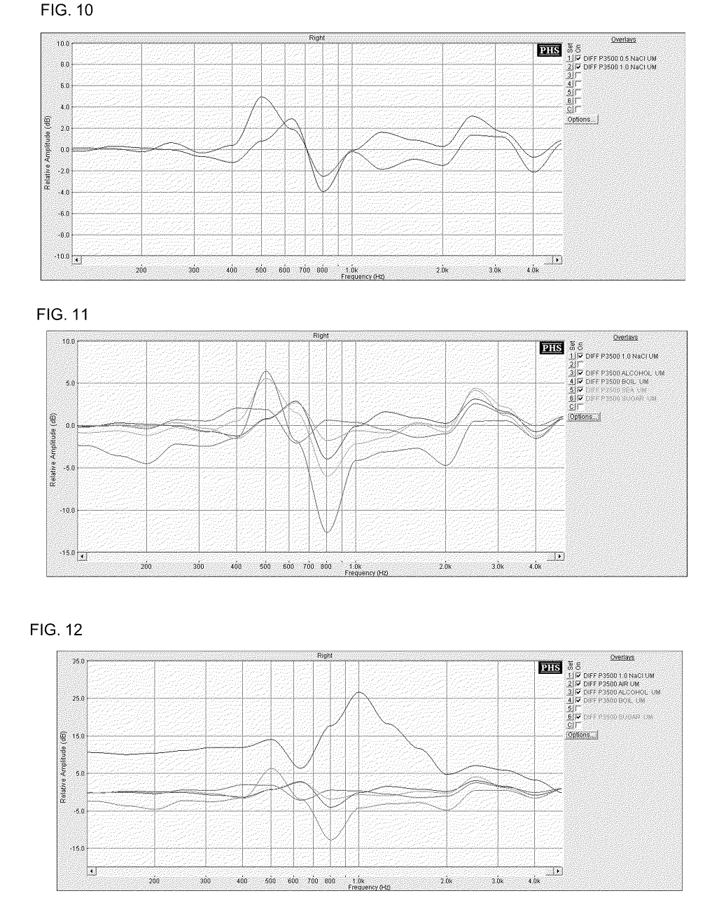

FIG. 12 illustrates the relative differences of amplitude of 1 gram of NaCl solution, air, alcohol, baby oil, and 1 gram sugar in 100 ml, at 0.35 bar gauge pressure;

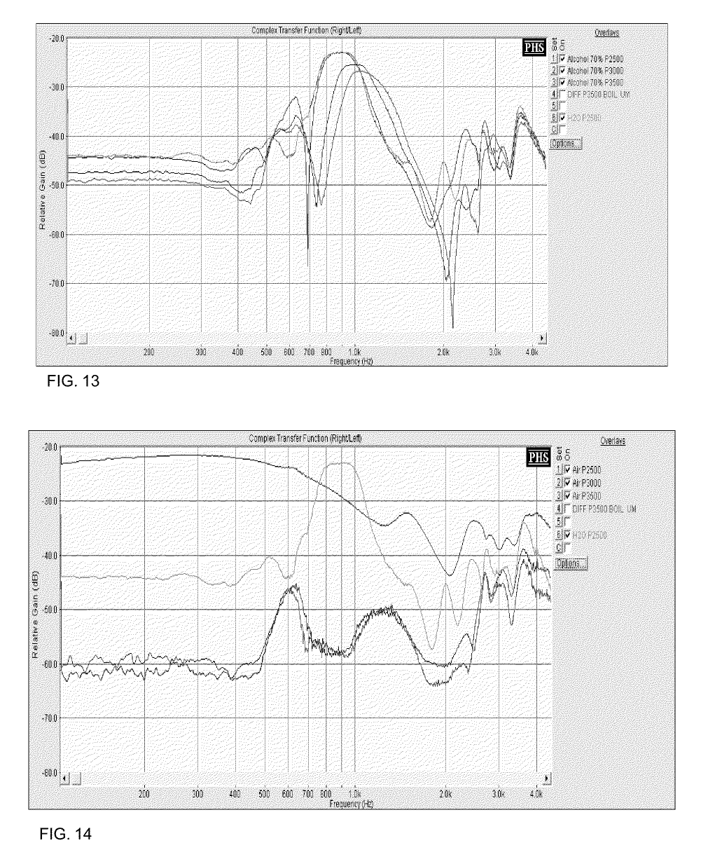

FIG. 13 illustrates the complex transfer function between the upstream and downstream microphone for alcohol at three pressures 0.25 bar, 0.3 bar, and 0.35 bar;

FIG. 14 illustrates the complex transfer function between the upstream and downstream microphone for air at three pressures 0.25 bar, 0.3 bar, and 0.35 bar;

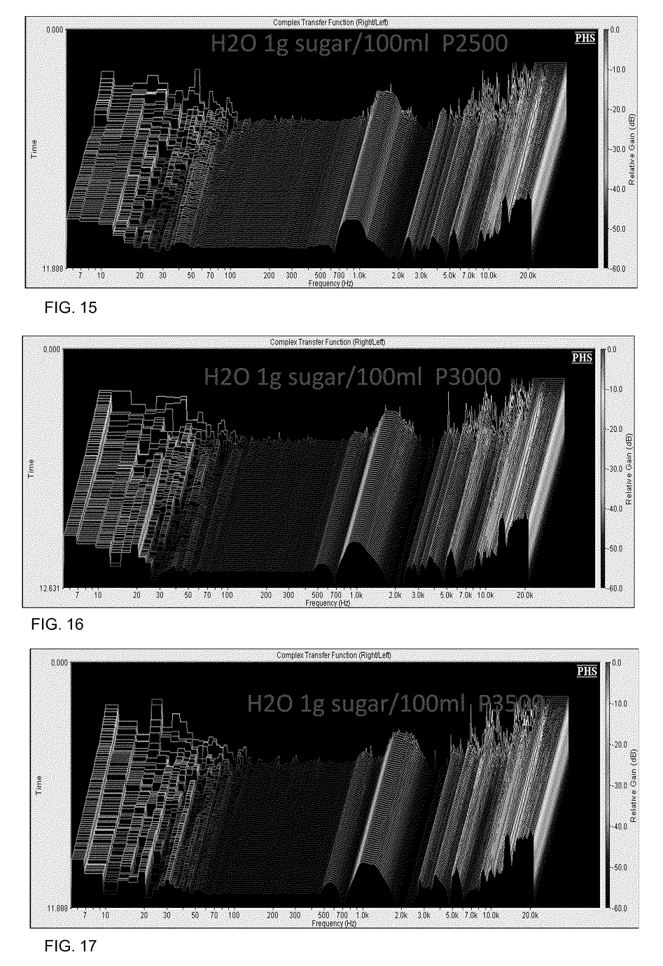

FIG. 15-17 illustrate spectrograms for 1 gram of sucrose in 100 ml water at three pressures 0.25 bar, 0.3 bar, and 0.35 bar;

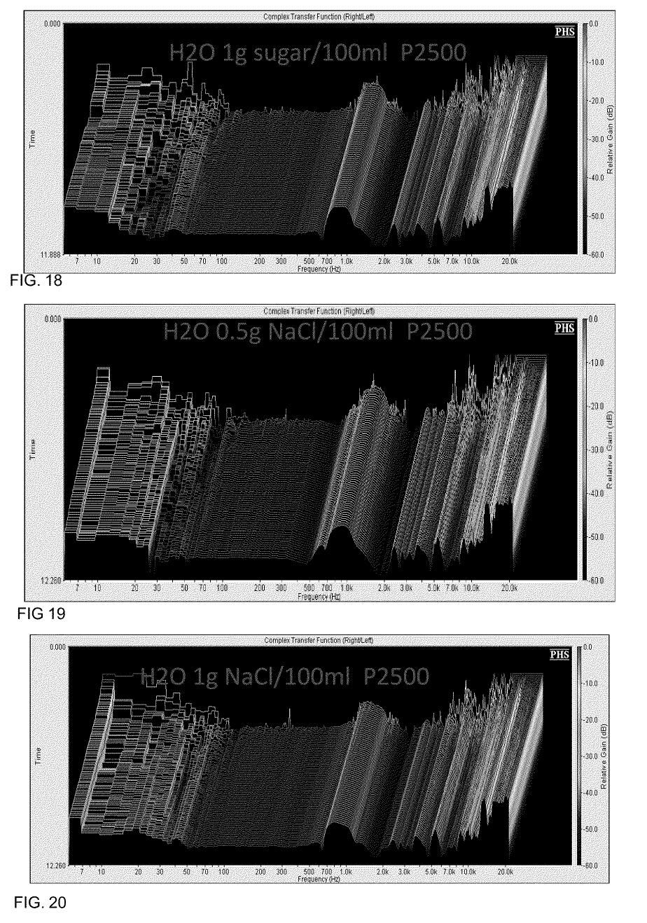

FIG. 18-20 illustrate spectrograms for 1 gram of sucrose, 0.5 grams of NaCl in 100 ml, and 1 gram of NaCl in 100 ml at the same pressure 0.25 bar gauge; and

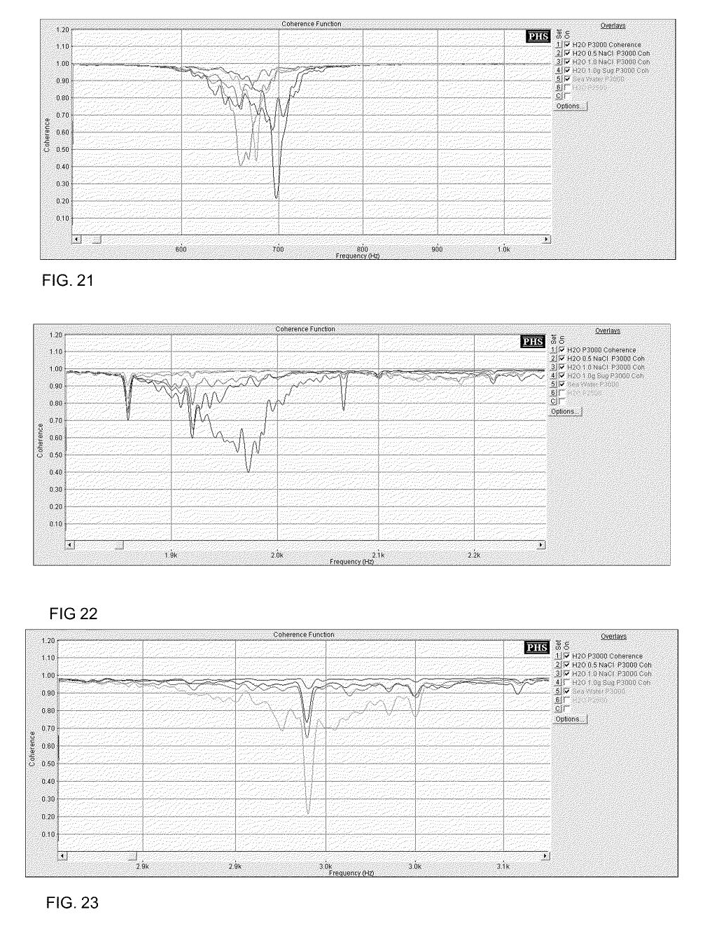

FIGS. 21-23 illustrate NaCl various concentrations and sucrose various concentrations, where the coherence between the upstream and downstream microphone are illustrated, where differences in concentration can be clearly seen and differences in substance can be seen, and where the concentration differences occur in a a frequency bands and where the substance differences occur in the same and other frequency bands.

FIG. 4A illustrates an acoustic sensor 100 in accordance with at least one exemplary embodiment, where a receiver 110, emits 150 sound 160 through a medium 140 that can be traveling 147, part of the sound is reflected 170, where the incident and reflected sound is measured by an upstream microphone 183, the transmitted 190 sound 195 is measured by a downstream microphone 185 and can also be measured by a second downstream microphone 120. Note that additional microphones (e.g., 120, 187 can be added and used).

FIG. 21 illustrates the use of coherence between an upstream and downstream microphone to distinguish substances (e.g., NaCl and sucrose concentrations in water).

FIG. 23 illustrate using coherence to determine the concentration of a substance in water, for example NaCl.

Where applicable, the present embodiments of the invention can be realized in hardware, software or a combination of hardware and software. Any kind of computer system or other apparatus adapted for carrying out the methods described herein are suitable. A typical combination of hardware and software can be a mobile communications device with a computer program that, when being loaded and executed, can control the mobile communications device such that it carries out the methods described herein. Portions of the present method and system may also be embedded in a computer program product, which comprises all the features enabling the implementation of the methods described herein and which when loaded in a computer system, is able to carry out these methods.

While the preferred embodiments of the invention have been illustrated and described, it will be clear that the embodiments of the invention are not so limited. Numerous modifications, changes, variations, substitutions and equivalents will occur to those skilled in the art without departing from the spirit and scope of the present embodiments of the invention as defined by the appended claims.

What is claimed is:

1. An acoustic sensor comprising:
a first receiver configured to emit a first acoustic signal;
a first microphone configured to measure a first measurement signal;
a second microphone configured to measure a second measurement signal;
a chamber; and
a first medium, where the first medium is in the chamber, where at least a first portion of the first acoustic signal passes through the medium, where the first measurement signal includes at least a second portion of the first acoustic signal, where the second measurement signal includes at least a third portion of the first acoustic signal, where the second and third portions are compared to stored identification data, where a match of the second and third portions with the stored identification data produces an identification for the first medium.

2. A medical diagnostic device comprising:
a first receiver configured to emit a first acoustic signal;
a first microphone configured to measure a first measurement signal;
a second microphone configured to measure a second measurement signal; and
a finger where at least a first portion of the first acoustic signal passes through the finger, where the first measurement signal includes at least a second portion of the first acoustic signal, where the second measurement signal includes at least a third portion of the first acoustic signal, where the second and third portions are compared to stored identification data, where a match of the second and third portions with the stored identification data produces an identification for the blood sugar levels in the finger.
